SEQUENCE LISTING (27 sequences)

SEQ ID NO: 1 - protein, 455 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKFSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 2 - protein, 460 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKFSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 3 - protein, 455 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGFISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 4 - protein, 460 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGFISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 5 - protein, 455 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGFFSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 6 - protein, 460 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGFFSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 7 - protein, 455 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGFYSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 8 - protein, 460 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGFYSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 9 - protein, 455 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGYFSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 10 - protein, 460 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGYFSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 11 - protein, 456 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKFSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 461 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKFSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 456 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGFISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 461 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGFISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 15 - protein, 456 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGFYSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 461 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGFYSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 456 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGFYSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 456 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKIQITQLGLQQSTITFWQITLEDEGCYMCLFNTFGFGFYSGTACLTVYVQPIVSLHYKFSEDHLQITCSATARPAPMVFWKVPRSGIEQSTVTLSHPQGTTSVTSILHIKDPKNQVGKEVICQVLHLGTVTDFKQTVNKGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 19 - protein, 456 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGFFSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 461 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGFFSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 456 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGYFSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 461 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGYFSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 456 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGYYSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLGSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 455 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 25 - protein, 460 aa, synthetic construct
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 26 - protein, 201 aa, Homo sapiens
QVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNK

SEQ ID NO: 27 - protein, 278 aa, Homo sapiens
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGKISGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGYWFSVPLLLSIVSLVILLVLISILLYWKRHRNQDRGELSQGVQKMT

(19) United States
(12) Patent Application Publication
(10) Pub. No.: US 2025/0026805 A1
(43) Pub. Date: Jan. 23, 2025

(54) NOVEL PROTEINS

(71) Applicant: DUCENTIS BIOTHERAPEUTICS LIMITED, Bristol (GB)

(72) Inventors: Rebecca ASHFIELD, Bristol (GB); Philip HUXLEY, Bristol (GB)

(73) Assignee: DUCENTIS BIOTHERAPEUTICS LIMITED, Bristol (GB)

(21) Appl. No.: 18/707,321

(22) PCT Filed: Nov. 3, 2022
(86) PCT No.: PCT/GB2022/052764

§ 371 Date: May 3, 2024

(30) Foreign Application Priority Data
Nov. 3, 2021 (GB) 2115803.5

(51) Int. Cl. C07K 14/705; A61K 38/17

(52) U.S. Cl. CPC C07K 14/70503; A61K 38/1774; C07K 2319/30

(57) ABSTRACT

The invention relates generally to mutant CD200 proteins which bind with greater affinity to the CD200 receptor than wild-type CD200, in particular the invention relates to a mutated CD200 protein comprising specific mutations at amino acid residue position 130 and/or 131. This invention also relates to a fusion protein comprising the protein as defined herein fused to a non-CD200 protein portion directly or via an optional linker portion, a pharmaceutical composition comprising the protein as defined herein and uses thereof.

### FIELD OF THE INVENTION

The invention relates generally to mutant CD200 proteins which bind with greater affinity to the CD200 receptor than wild-type CD200, in particular the invention relates to a mutated CD200 protein comprising specific mutations at amino acid residue position 130 and/or 131. This invention also relates to a fusion protein comprising the protein as defined herein fused to a non-CD200 protein portion directly or via an optional linker portion, a pharmaceutical composition comprising the protein as defined herein and uses thereof.

### BACKGROUND OF THE INVENTION

Autoimmune diseases are the second leading cause of chronic illness globally and in the U.S they are the leading cause of morbidity in women. According to a 2008 international survey, chronically ill patients in the U.S. as compared with those in other countries are more likely to do without proper care due to the burden of cost (Schoen, C. et al., (2008) Health Affairs Web Exclusive, w1-w16). Additionally, these patients are more likely to experience the highest rates of medical errors, problems with coordination of care, and high out-of-pocket health care costs.

Currently, the American Autoimmune Related Disease Association (AARDA) estimates that 50 million Americans have an autoimmune disease. Epidemiological data are lacking to determine the full direct and indirect cost to the overall health care system due to autoimmune disease. However, in 2001, the National Institutes of Allergy and Infectious Diseases (NIAID) Director Dr. Anthony Fauci estimated that annual autoimmune disease treatment costs were greater than $100 billion. While $100 billion is a staggering figure, it is likely a vast understatement of the true costs of autoimmune disease as the annual costs of only seven of the 100+ known autoimmune diseases, Crohn's disease, ulcerative colitis, systemic lupus erythematosus (SLE), multiple sclerosis (MS), rheumatoid arthritis (RA), psoriasis, and scleroderma, are estimated through epidemiological studies to total from $51.8-$70.6 billion annually. Furthermore, these estimates overlook the cost of immunosuppressive therapy during transplantation.

Autoimmune diseases are chronic conditions with no cure, which arise when the immune system decides that healthy cells are foreign and attacks them. Depending on the type, an autoimmune disease can affect one or many different types of body tissue and can cause abnormal organ growth and changes in organ function. The normal regulation of the immune system is largely due to receptor/ligand pairs that includes proteins that are expressed by cells involved in an immune response. However, these receptor/ligand pairs are often included in signalling cascades which contribute to the pathology of autoimmune disease.

OX-2 membrane glycoprotein, also named CD200 (Cluster of Differentiation 200), is a human protein encoded by the CD200 gene which is expressed in a variety of cell types (Barclay, A. N. (1981) Immunology 44, 727) and has a high degree of homology to molecules of the immunoglobulin gene family. The protein encoded by this gene is a type-1 membrane glycoprotein which contains two immunoglobulin domains and binds to the CD200 receptor (CD200R).

CD200R is expressed on myeloid cells (monocytes, macrophages, dendritic cells and eosinophils) and T cells (Wright, et al., (2000), Immunity 12, 233-242; Wright, et al., (2003), J. Immunol, 171, 3034-3046).

Engagement of CD200 with CD200R delivers an inhibitory signal to myeloid and T-cells, thus exerting an immunosuppressive effect on both the innate and adaptive arms of the immune system (Rahim S. A., (2005) AIDS, 19, 1907-1925; Shiratori, I., (2005) J. Immunol, 175, 4441-4449; Misstear, K., et al., (2012), Journal of Virology, 86 (11), 6246-6257).

CD200R agonists have been shown to reduce pathology in a wide range of murine disease models, for example arthritis (Gorczynski, et al., (2001) Clin. Immunol. 101, 328-34; Gorczynski, et al., (2002) Clin. Immunol. 104, 256-264), graft rejection (Gorczynski, et al., (2002) Transplantation 73, 1948-1953), failed pregnancy (Gorczynski, et al., (2002) Am. J. Reprod. Immunol., 48, 18-26), contact hypersensitivity (Rosenblum, et al., (2004) Blood 103, 2691-8), influenza induced lung inflammation (Snelgrove, et al., (2008) Nat. Immunol., 9, 1074-1083) and HSV-induced inflammatory lesions (Sarangi, et al., (2009) Clin. Immunol. 131, 31-40).

Additionally, CD200^{-/-} mice challenged with influenza virus developed more severe disease, which was associated with increased lung infiltration and lung endothelium damage, compared with wildtype controls (Rygiel. T. P., et al. (2009) J. Immunol. 183 (3), 1990-1996). CD200^{-/-} mice did develop immune responses that could control viral load, suggesting that the severe disease was caused by poor control of the immune response as opposed to the beneficial antiviral immune response. Disease could be prevented by T-cell depletion before viral challenge, despite the dramatically increased viral load that resulted. Rygiel. T. P., et al. (2009) concluded that T cells are essential for the manifestation of disease symptoms during influenza infection, and that lack of down-modulating CD200-CD200R signalling, rather than viral load, increases immune pathology.

Profiling studies have shown that hCD200 expression is down regulated in diverse patient populations, such as patients with multiple sclerosis (Koning, et al., (2007) Ann. Neurol. 62, 504-514), asthma exacerbation (Aoki, et al., (2009) Clin. Exp. Allergy 39, 213-221), Alzheimer's disease (Walker, et al., (2009) Exp. Neurol. 215, 5-19), primary hypertrophic osteoarthropathy (Ren, et al., (2013) Rheumatol. Int. 33 (10), 2509-2512), failed pregnancy (Clark (2009) Am. J. Reprod. Immunol. 61, 75-84) and lichen planopilaris (hair loss) (Harries, et al., (2013) J. Pathol. 231 (2), 236-247).

Agonist CD200 proteins are disclosed in, for example, WO 2000/061171 and WO 2008/089022. The literature describes the use of wild-type CD200 molecules to modulate immune cell function. The invention relates to mutant CD200 proteins which bind with greater affinity to the CD200 receptor than wild-type CD200.

Therapeutic intervention with molecules that modulate the CD200 pathway therefore offer a means of controlling exaggerated or unwanted immune responses and reducing pathology in patients suffering from chronic or intermittent (flare-up) autoimmune disease.

There is therefore a need to provide improved clinical efficacy at lower doses which will overcome the problems associated with currently available treatments to autoimmune diseases.

### SUMMARY OF THE INVENTION

According to a first aspect of the invention, there is provided a mutated CD200 protein comprising the following mutations:
(i) K130F; or
(ii) I131F; or
(iii) K130F and I131F; or
(iv) K130F and I131Y; or
(v) K130Y and I131F.

According to a second aspect of the invention, there is provided a fusion protein comprising the protein as defined herein, fused to a non-CD200 portion directly or via an optional linker portion.

According to a yet further aspect of the invention, there is provided a polynucleotide encoding a protein or a fusion protein as defined herein.

According to a further aspect of the invention, there is provided a pharmaceutical composition comprising the protein, polypeptide or fusion protein as defined herein.

According to a further aspect of the invention, there is provided the protein, fusion protein or pharmaceutical composition as defined herein for use in the treatment of autoimmune disease, an allergic disease, neurodegeneration or neuropathic pain.

### BRIEF DESCRIPTION OF THE FIGURES

FIG. 1: Surface Plasmon Resonance (SPR) sensorgrams illustrating the binding response and off rates of high affinity CD200-Fc (Panels A-F) and wild-type CD200-Fc (panel G) fusion molecules binding to human CD200 receptor at 25° C.

FIGS. 2 to 7: Bar charts demonstrating inhibition of IL-6 secretion following LPS stimulation of CD200R expressing U937 cells.

### DETAILED DESCRIPTION OF THE INVENTION

According to a first aspect of the invention, there is provided a mutated CD200 protein comprising the following mutations:
(i) K130F; or
(ii) I131F; or
(iii) K130F and I131F; or
(iv) K130F and I131Y; or
(v) K130Y and I131F.

According to a further aspect of the invention, there is provided a polypeptide comprising a mutated CD200 protein comprising at least 90% identity to: QVQVVTQDEREQLYTPASLKCSLQNAQEALIVTWQKKKAVSPENMVTFSENHGVVIQPAYK DKINITQLGLQNSTITFWNITLEDEGCYMCLFNTFGFGKISGTACLTVYVQPIVSLHYKFSEDH LNITCSATARPAPMVFWKVPRSGIENSTVTLSHPNGTTSVTSILHIKDPKNQVGKEVICQVLH LGTVTDFKQTVNK (SEQ ID NO: 26) with the following mutations at positions 130 and/or 131:
(i) K130F; or
(ii) I131F; or
(iii) K130F and I131F; or
(iv) K130F and I131Y; or
(v) K130Y and I131F.

It will be appreciated that the polypeptide sequence of SEQ ID NO: 26 relates to the wild-type polypeptide sequence of the extracellular domain of CD200.

In one embodiment, the polypeptide comprises at least 90% identity to the amino acid sequence of: MERLVIRMPFSHLSTYSLVWVMAAVVLCTAQVQVVTQDEREQLYTPASLKCSLQNAQEALI VTWQKKKAVSPENMVTFSENHGVVIQPAYKDKINITQLGLQNSTITFWNITLEDEGCYMCLF NTFGFGKISGTACLTVYVQPIVSLHYKFSEDHLNITCSATARPAPMVFWKVPRSGIENSTVTL SHPNGTTSVTSILHIKDPKNQVGKEVICQVLHLGTVTDFKQTVNKGYWFSVPLLLSIVSLVILL VLISILLYWKRHRNQDRGELSQGVQKMT (SEQ ID NO: 27) with the following mutations at positions 130 and/or 131:
(i) K130F; or
(ii) I131F; or
(iii) K130F and |131F; or
(iv) K130F and I131Y; or
(v) K130Y and I131F.

It will be appreciated that the polypeptide sequence of SEQ ID NO: 27 relates to the full length wild-type polypeptide sequence of CD200.

The inventors have found that mutations of CD200 at the specific amino acid residues (i) to (v) produce a mutant CD200 with increased binding affinity to the CD200 receptor (CD200R). Additionally, the inventors have found that optimal efficacy is obtained with molecules that demonstrate high affinity binding combined with low residence times on the CD200 receptor, such as those which do not exceed 3000 seconds. Additionally, the mutated CD200 proteins as described herein have significant benefits, in particular in respect to providing treatment with greater clinical efficacy and at lower doses.

The term “CD200 protein” as used herein, refers to wild-type CD200 protein.

The term “wild-type” as used herein, refers to proteins, peptides, amino acid and nucleotide sequences which are present in nature. For example, the term “wild-type CD200 protein” as used herein, refers to any full-length isoform of CD200 (UNIPROT P41217 OX2G_HUMAN) or any portion thereof (including naturally occurring protein polymorphisms) which binds to the CD200 receptor. CD200 protein is also known as OX-2 membrane glycoprotein.

Wild-type CD200 is a cell surface protein, having an N-terminal extracellular domain, and short transmembrane and cytoplasmic domains. The extracellular domain binds to target receptors such as the CD200 receptor. In one embodiment, the CD200 protein is the extracellular domain of CD200, or any portion thereof, which binds to the CD200 receptor.

The term “position” as used herein, refers to the residue number in an amino acid sequence where 1 is the first translated amino acid.

The term “mutated” or “mutation” as used herein, refers to proteins, peptides, amino acid and nucleotide sequences which have undergone a change in their form from the wild-type equivalent to become a mutant. For example, a mutated or mutant protein may have undergone a change in the amino acid and/or nucleotide sequence when compared to the corresponding wild-type sequence, such a change may also be referred to as a mutation.

The term “mutated CD200 protein” as used herein, refers to full length CD200 protein or any portions thereof, which binds to the CD200 receptor, comprising a mutated amino acid residue or multiple mutated amino acid residues in the amino acid sequence so that it is similar but no longer identical to the wild-type CD200 protein.

In one embodiment, the mutated CD200 protein may be made synthetically or recombinantly. In a further embodiment, the mutated CD200 protein may be made synthetically. In an alternative embodiment, the mutated CD200 protein may be made recombinantly.

In one embodiment, the mutated CD200 protein binds to the CD200 receptor with greater affinity than wild-type CD200.

In one embodiment, the mutated CD200 protein may comprise a biologically or chemically active non-CD200 component therein or attached thereto.

In one embodiment, the mutated CD200 protein may be soluble (i.e. circulating) or bound to a surface. In a further embodiment, the mutated CD200 protein is soluble. In an alternative embodiment, the mutated CD200 protein is bound to a surface.

In one embodiment, the mutated CD200 protein may include the entire extracellular domain of CD200 or portions thereof. In further embodiments, the mutated CD200 protein includes a signal sequence. It will be appreciated that secreted proteins comprise a number of amino acids at the N-terminus which make up a signal sequence which may be cleaved prior to secretion. Thus, in certain embodiments, the mutated CD200 protein includes a signal sequence at the N-terminus which is cleaved prior to secretion from the producing cell. In further embodiments, the signal sequence may be cleaved at any position selected from amino acids 16-35 of wild-type CD200 protein. In one embodiment the signal sequence comprises the first 28 amino acids of wild-type CD200 protein. In an alternative embodiment, the signal sequence comprises the first 29 amino acids of wild-type CD200 protein. In a further alternative embodiment, the signal sequence comprises the first 30 amino acids of wild-type CD200 protein. In a yet further alternative embodiment, the signal sequence comprises the first 31 amino acids of wild-type CD200 protein. In a yet further alternative embodiment, the signal sequence comprises the first 32 amino acids of wild-type CD200 protein. Therefore, in certain embodiments, the mutated CD200 protein comprises a sequence as defined herein, where the amino acids which comprise the signal sequence are absent. For example, where amino acids 1-30 of wild-type CD200 protein are absent and the mutated CD200 protein comprises a sequence corresponding to amino acids 31-232 of any sequence defined herein.

The term “portion” as used herein with reference to proteins, peptides and amino acid and nucleotide sequences, refers to fragments and derivatives that are functional, i.e. bind to their target.

The term “fragment” as used herein refers to a part of a protein, peptide, amino acid or nucleotide sequence that recognises and binds its target, such as a receptor.

The term “derivatives of” and “mutant” as used herein, refer to a protein, peptide, amino acid or nucleotide sequence that shares at least 70% (such as 75%, 80%, 85%, 90%, 95% or 99%) sequence similarity with and functions like the wild-type equivalent. Thus, a mutant may be a derivative of a wild-type equivalent.

The term “amino acid residue” as used herein, refers to a monomeric unit in a polymeric chain, i.e. a single amino acid in a protein.

The term “at least 90% identity” as used herein, refers to a sequence which shares sequence identity or sequence homology. Suitable examples include 91%, 92%, 93%, 94%, 95%, 96%, 97%, 98%, 99% or 100% identity.

In one embodiment, the protein additionally comprises one or more mutations present in the amino acid sequence, for example 1-15 mutations.

In one embodiment, the mutated CD200 protein comprises a single substitution mutation of K130F. Specific examples of mutated proteins comprising this single substitution mutation are described herein as DS-175, DS-161, DS-174 and DS-213.

In an alternative embodiment, the mutated CD200 protein comprises a single substitution mutation of I131F. Specific examples of mutated proteins comprising this single substitution mutation are described herein as DS-215, DS-162, DS-216 and DS-214.

In an alternative embodiment, the mutated CD200 protein comprises a double substitution mutation of K130F and I131F. Specific examples of mutated proteins comprising this double substitution mutation are described herein as DS-217, DS-150, DS-218 and DS-220.

In an alternative embodiment, the mutated CD200 protein comprises a double substitution mutation of K130F and I131Y. Specific examples of mutated proteins comprising this double substitution mutation are described herein as DS-164, DS-151, DS-163, DS-219, DS-167 and DS-165.

In an alternative embodiment, the mutated CD200 protein comprises a double substitution mutation of K130Y and I131F. Specific examples of mutated proteins comprising this double substitution mutation are described herein as DS-221, DS-149, DS-222 and DS-223.

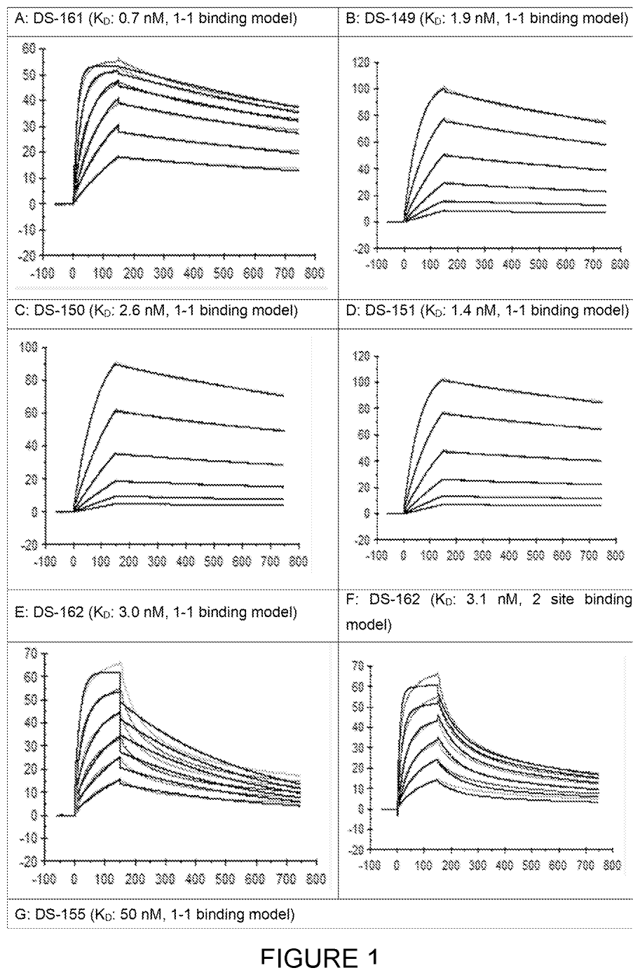

As presented in FIG. 1 and Table 4, the mutated CD200 proteins of the invention bind more tightly to the CD200 receptor and exhibit longer residence time on the receptor than wild-type CD200 protein.

### Fusion Protein

According to a second aspect of the invention, there is provided a fusion protein comprising the protein as defined herein fused to a non-CD200 portion directly or via an optional linker portion.

For example, the linker portion is a peptide comprising between 1 and 15 amino acids, e.g., 1, 2, 3, 4, 5, 6, 7, 8, 9, 10, 11, 12, 13, 14 or 15 amino acids.

The term “fusion protein” as used herein, refers to one or more amino acid sequences, peptides and/or proteins joined together using methods well known in the art and as described in, for example U.S. Pat. Nos. 5,434,131 and 5,637,481. The joined amino acid sequences, peptides or proteins thereby form one fusion protein.

In one embodiment, the protein herein is fused at the C-terminus to a non-CD200 portion directly or via an optional linker portion.

The term “non-CD200 portion” as used herein, refers to any molecule, peptide or protein that does not specifically bind to the CD200 receptor and does not interfere with the binding of the mutated CD200 protein to its target. Examples include, but are not limited to, an immunoglobulin (Ig) constant region or a portion thereof; or fusion proteins where the non-CD200 portion is a synthetic molecule, for example PEG.

In one embodiment, said non-CD200 portion is an antibody or fragment thereof. In a further embodiment, said non-CD200 portion is an Fc fragment. Therefore, the mutated CD200 fusion protein as described herein may also be called a mutant CD200-Fc. In a further embodiment, the Fc fragment is mammalian derived, such as derived from a human or monkey, such as human C (gamma) 1 which includes the hinge, CH2 and CH3 regions. The Fc fragment provides the advantage of increasing the serum half-life of the mutated CD200 proteins of the invention, and additionally increases binding avidity and enables agonistic signalling, by dimerising the CD200 proteins. It will be understood by one skilled in the art that the Fc region may be mutated to reduce its effector functions (see for example, U.S. Pat. Nos. 5,637,481 and 6,132,992).

In some embodiments, the human Fc domains include mutations to eliminate glycosylation and/or to reduce Fc-gamma receptor binding. In some embodiments, the human Fc domains comprise the mutation N297Q, N297A, or N297G; in some embodiments the human Fc domains comprise a mutation at position 234 and/or 235, for example L235E, or L234A and L235A (in IgG1), or F234A and L235A (in IgG4); in some embodiments the human Fc domains are IgG2 Fc domains that comprise the mutations V234A, G237A, P238S, H268Q/A, V309L, A330S, or P331S, or a combination thereof (all according to Kabat, EU numbering). In some embodiments, the human Fc domains each comprise human IgG1 constant region mutations L234A/L235A (“LALA”) or human IgG1 constant region mutations L234A/L235A/P329G (“LALAPG”). Additional examples of engineered human Fc domains are known to those skilled in the art. Examples of Ig heavy chain constant region amino acids in which mutations in at least one amino acid leads to reduced Fc function include, but are not limited to, mutations in amino acid 228, 233, 234, 235, 236, 237, 239, 252, 254, 256, 265, 270, 297, 318, 320, 322, 327, 329, 330, and 331 of the heavy constant region (according to Kabat, EU numbering). Examples of combinations of mutated amino acids are also known in the art, such as, but not limited to a combination of mutations in amino acids 234, 235, and 331, such as 234, 235, and 329, such as L234F, L235E, and P331S or a combination of amino acids 318, 320, and 322, such as E318A, K320A, and K322A.

Further examples of engineered Fc domains include F243L/R292P/Y300L/V305l/P396 IgG1; S239D/1332E IgG1; S239D/1332E/A330L IgG1; S298A/E333A/K334A; in one heavy chain, L234Y/L235Q/G236W/S239M/H268D/D270E/S298A IgG1, and in the opposing heavy chain, D270E/K326D, A330M/K334E IgG; G236A/S239D/1332E IgG1; K326W/E333S IgG1; S267E/H268F/S324T IgG1; E345R/E430G/S440Y IgG1; N297A or N297Q or N297G IgG1; L235E IgG1; L234A/L235A IgG1; F234A/L235A IgG4; H268Q/V309L/A330S/P331S IgG2; V234A/G237A/P238S/H268A/V309L/A330S/P331S IgG2; M252Y/S254T/T256E IgG1; M428L/N434S IgG1; S267E/L328F IgG1; N325S/L328F IgG1, and the like. In some embodiments, the engineered Fc domain comprises one or more substitutions selected from the group consisting of N297A IgG1, N297Q IgG1, and S228P IgG4.

In one aspect, polypeptides of the present disclosure comprising an Fc variant exhibit decreased affinities to an Fc receptor, e.g., FcγRI, FcγRIIA, FcγRIIIA, relative to an unmodified antibody. In one aspect, polypeptides comprising an Fc variant exhibit affinity for the Fc receptor that is at least 95%, at least 90%, at least 80%, at least 70%, at least 60%, at least 50%, at least 40%, at least 30%, at least 20%, at least 10%, least 5%, or at least 1% less than a than that of a wild type polypeptide. In one aspect, polypeptides comprising an Fc variant of the present disclosure exhibit, greater than 700-fold reduction in Fcγ binding, or greater than 3,500-fold reduction in Fcγ binding.

In some embodiments, the antibody or antigen-binding fragment thereof comprises a variant Fc region of IgG1, IgG2, IgG3, IgG4, IgA, IgE, or IgM. In certain embodiments the antibody is an aglycosylated antibody with reduced effector functions. In certain embodiments, the variant Fc region of IgG1 comprises (a) an amino acid substitution at position Leu234 with alanine; (b) an amino acid substitution at position Leu235 with alanine; (c) an amino acid substitution at position Pro329 with glycine or arginine; (d) Asn297 with alanine; I Asn297 with glutamine; (f) Asn297 with glycine; or (g) any combination of (a) to (f). In certain embodiments, the variant Fc region of IgG2 comprises (g) an amino acid substitution at position Ser228 with proline; (h) an amino acid substitution at position Pro329 with glycine or arginine; or (i) both (g) and (h). In certain embodiments, the variant Fc region of IgG4 comprises (j) an amino acid substitution at position Ser228 with proline; (k) an amino acid substitution at position Leu235 with alanine or glutamate; (l) an amino acid substitution at position Pro329 with glycine or arginine; or (m) any combination of (j) to (l).

In one embodiment, the Fc fragment is or is derived from human IgG2 or human IgG4.

In a further embodiment, the non-CD200 portion is an antibody Fc fragment which comprises mutation of one or more amino acid residue(s). Thus, in a further embodiment, the Fc fragment is a S228P derivative of human IgG4.

In one embodiment, the fusion protein is an Fc fusion protein formed by direct fusion of amino acid Glycine 232 of CD200 to amino acid 1 of the Fc hinge region.

In an alternative embodiment, the fusion protein is an Fc fusion protein formed by direct fusion of amino acid Glycine 232 of CD200 to amino acid 6 of the Fc hinge region.
(in this case the first 5 amino acids of the Fc hinge are deleted).

For the purpose of this description, the term “position” as used herein with respect to a non-CD200 portion when said non-CD200 portion is an Fc fragment, refers to the residue number in an amino acid sequence according to the EU numbering system. Therefore, it will be appreciated that a residue position as quoted herein for an amino acid of an Fc fragment relates to its position according to the EU numbering system. It will be further appreciated that other numbering systems developed for the numbering of residues in Fc fragment sequences, such as Kabat, Aho, IMGT, Chothia and Martin (enhanced Chothia), may alternatively be utilised. For example, when referring herein to the Fc hinge region, it is intended to refer to the region of the Fc domain which starts at amino acid position 1 as defined by IMGT numbering (https://www.imgt.org/IMGTScientificChart/Numbering/Hu_IGHGnber.html), or amino acid position 216 as defined by the EU numbering system.

Specific examples of fusion proteins of the invention are exemplified in Table 1 as SEQ ID NOS: 1 to 22.

In one embodiment, the fusion protein is selected from any one of SEQ ID NOS: 1 to 22. In a further embodiment, the fusion protein is selected from any one of SEQ ID NOS: 1 to 16 and 19 to 22.

| Mutant | | | CD200-Fc | |
|---|---|---|---|---|
| Number | CD200 Mutation | Fc | junction | Full Fusion Protein Sequence (including CD200 and Fc) |
| DS-215 | I131F | IgG2 | CD200 | MERLVIRMPFSHLSTYSLVWVMAAVVLCTAQVQVVTQDEREQLYTPASLKCSLQNAQEALI |
| | | | amino acid | VTWQKKKAVSPENMVTFSENHGVVIQPAYKDKINITQLGLQNSTITFWNITLEDEGCYMCLF |
| | | | 232 to | NTFGFGKFSGTACLTVYVQPIVSLHYKFSEDHLNITCSATARPAPMVFWKVPRSGIENSTVT |
| | | | amino acid | LSHPNGTTSVTSILHIKDPKNQVGKEVICQVLHLGTVTDFKQTVNKGVECPPCPAPPVAGPS |
| | | | 6 of Fc | VFLFPPKPKDTLMISRTPEVTCVVVDVSHEDPEVQFNWYVDGVEVHNAKTKPREEQFNST |
| | | | hinge | FRVVSVLTVVHQDWLNGKEYKCKVSNKGLPAPIEKTISKTKGQPREPQVYTLPPSREEMTK |
| | | | | NQVSLTCLVKGFYPSDISVEWESNGQPENNYKTTPPMLDSDGSFFLYSKLTVDKSRWQQG |
| | | | | NVFSCSVMHEALHNHYTQKSLSLSPGK (SEQ ID NO: 1) |
| DS-162 | I131F | IgG2 | CD200 | MERLVIRMPFSHLSTYSLVWVMAAVVLCTAQVQVVTQDEREQLYTPASLKCSLQNAQEALI |
| | | | amino acid | VTWQKKKAVSPENMVTFSENHGVVIQPAYKDKINITQLGLQNSTITFWNITLEDEGCYMCLF |
| | | | 232 to | NTFGFGKFSGTACLTVYVQPIVSLHYKFSEDHLNITCSATARPAPMVFWKVPRSGIENSTVT |
| | | | amino acid | LSHPNGTTSVTSILHIKDPKNQVGKEVICQVLHLGTVTDFKQTVNKGERKCCVECPPCPAPP |
| | | | 1 of Fc | VAGPSVFLFPPKPKDTLMISRTPEVTCVVVDVSHEDPEVQFNWYVDGVEVHNAKTKPREE |
| | | | hinge | QFNSTFRVVSVLTVVHQDWLNGKEYKCKVSNKGLPAPIEKTISKTKGQPREPQVYTLPPSR |
| | | | | EEMTKNQVSLTCLVKGFYPSDISVEWESNGQPENNYKTTPPMLDSDGSFFLYSKLTVDKS |
| | | | | RWQQGNVFSCSVMHEALHNHYTQKSLSLSPGK (SEQ ID NO: 2) |
| DS-175 | K130F | IgG2 | CD200 | MERLVIRMPFSHLSTYSLVWVMAAVVLCTAQVQVVTQDEREQLYTPASLKCSLQNAQEALI |
| | | | amino acid | VTWQKKKAVSPENMVTFSENHGVVIQPAYKDKINITQLGLQNSTITFWNITLEDEGCYMCLF |
| | | | 232 to | NTFGFGFISGTACLTVYVQPIVSLHYKFSEDHLNITCSATARPAPMVFWKVPRSGIENSTVTL |
| | | | amino acid | SHPNGTTSVTSILHIKDPKNQVGKEVICQVLHLGTVTDFKQTVNKGVECPPCPAPPVAGPS |
| | | | 6 of Fc | VFLFPPKPKDTLMISRTPEVTCVVVDVSHEDPEVQFNWYVDGVEVHNAKTKPREEQFNST |
| | | | hinge | FRVVSVLTVVHQDWLNGKEYKCKVSNKGLPAPIEKTISKTKGQPREPQVYTLPPSREEMTK |
| | | | | NQVSLTCLVKGFYPSDISVEWESNGQPENNYKTTPPMLDSDGSFFLYSKLTVDKSRWQQG |
| | | | | NVFSCSVMHEALHNHYTQKSLSLSPGK (SEQ ID NO: 3) |
| DS-161 | K130F | IgG2 | CD200 | MERLVIRMPFSHLSTYSLVWVMAAVVLCTAQVQVVTQDEREQLYTPASLKCSLQNAQEALI |
| | | | amino acid | VTWQKKKAVSPENMVTFSENHGVVIQPAYKDKINITQLGLQNSTITFWNITLEDEGCYMCLF |
| | | | 232 to | NTFGFGFISGTACLTVYVQPIVSLHYKFSEDHLNITCSATARPAPMVFWKVPRSGIENSTVTL |
| | | | amino acid | SHPNGTTSVTSILHIKDPKNQVGKEVICQVLHLGTVTDFKQTVNKGERKCCVECPPCPAPP |
| | | | 1 of Fc | VAGPSVFLFPPKPKDTLMISRTPEVTCVVVDVSHEDPEVQFNWYVDGVEVHNAKTKPREE |
| | | | hinge | QFNSTFRVVSVLTVVHQDWLNGKEYKCKVSNKGLPAPIEKTISKTKGQPREPQVYTLPPSR |
| | | | | EEMTKNQVSLTCLVKGFYPSDISVEWESNGQPENNYKTTPPMLDSDGSFFLYSKLTVDKS |
| | | | | RWQQGNVFSCSVMHEALHNHYTQKSLSLSPGK (SEQ ID NO: 4) |
| DS-217 | K130F | IgG2 | CD200 | MERLVIRMPFSHLSTYSLVWVMAAVVLCTAQVQVVTQDEREQLYTPASLKCSLQNAQEALI |
| | I131F | | amino acid | VTWQKKKAVSPENMVTFSENHGVVIQPAYKDKINITQLGLQNSTITFWNITLEDEGCYMCLF |
| | | | 232 to | NTFGFGFFSGTACLTVYVQPIVSLHYKFSEDHLNITCSATARPAPMVFWKVPRSGIENSTVT |
| | | | amino acid | LSHPNGTTSVTSILHIKDPKNQVGKEVICQVLHLGTVTDFKQTVNKGVECPPCPAPPVAGPS |
| | | | 6 of Fc | VFLFPPKPKDTLMISRTPEVTCVVVDVSHEDPEVQFNWYVDGVEVHNAKTKPREEQFNST |
| | | | hinge | FRVVSVLTVVHQDWLNGKEYKCKVSNKGLPAPIEKTISKTKGQPREPQVYTLPPSREEMTK |
| | | | | NQVSLTCLVKGFYPSDISVEWESNGQPENNYKTTPPMLDSDGSFFLYSKLTVDKSRWQQG |
| | | | | NVFSCSVMHEALHNHYTQKSLSLSPGK (SEQ ID NO: 5) |
| DS-150 | K130F | IgG2 | CD200 | MERLVIRMPFSHLSTYSLVWVMAAVVLCTAQVQVVTQDEREQLYTPASLKCSLQNAQEALI |
| | I131F | | amino acid | VTWQKKKAVSPENMVTFSENHGVVIQPAYKDKINITQLGLQNSTITFWNITLEDEGCYMCLF |
| | | | 232 to | NTFGFGFFSGTACLTVYVQPIVSLHYKFSEDHLNITCSATARPAPMVFWKVPRSGIENSTVT |
| | | | amino acid | LSHPNGTTSVTSILHIKDPKNQVGKEVICQVLHLGTVTDFKQTVNKGERKCCVECPPCPAPP |
| | | | 1 of Fc | VAGPSVFLFPPKPKDTLMISRTPEVTCVVVDVSHEDPEVQFNWYVDGVEVHNAKTKPREE |
| | | | hinge | QFNSTFRVVSVLTVVHQDWLNGKEYKCKVSNKGLPAPIEKTISKTKGQPREPQVYTLPPSR |
| | | | | EEMTKNQVSLTCLVKGFYPSDISVEWESNGQPENNYKTTPPMLDSDGSFFLYSKLTVDKS |
| | | | | RWQQGNVFSCSVMHEALHNHYTQKSLSLSPGK (SEQ ID NO: 6) |
| DS-164 | K130F | IgG2 | CD200 | MERLVIRMPFSHLSTYSLVWVMAAVVLCTAQVQVVTQDEREQLYTPASLKCSLQNAQEALI |
| | I131Y | | amino acid | VTWQKKKAVSPENMVTFSENHGVVIQPAYKDKINITQLGLQNSTITFWNITLEDEGCYMCLF |
| | | | 232 to | NTFGFGFYSGTACLTVYVQPIVSLHYKFSEDHLNITCSATARPAPMVFWKVPRSGIENSTVT |
| | | | amino acid | LSHPNGTTSVTSILHIKDPKNQVGKEVICQVLHLGTVTDFKQTVNKGVECPPCPAPPVAGPS |
| | | | 6 of Fc | VFLFPPKPKDTLMISRTPEVTCVVVDVSHEDPEVQFNWYVDGVEVHNAKTKPREEQFNST |
| | | | hinge | FRVVSVLTVVHQDWLNGKEYKCKVSNKGLPAPIEKTISKTKGQPREPQVYTLPPSREEMTK |
| | | | | NQVSLTCLVKGFYPSDISVEWESNGQPENNYKTTPPMLDSDGSFFLYSKLTVDKSRWQQG |
| | | | | NVFSCSVMHEALHNHYTQKSLSLSPGK (SEQ ID NO: 7) |
| DS-151 | K130F | IgG2 | CD200 | MERLVIRMPFSHLSTYSLVWVMAAVVLCTAQVQVVTQDEREQLYTPASLKCSLQNAQEALI |
| | I131Y | | amino acid | VTWQKKKAVSPENMVTFSENHGVVIQPAYKDKINITQLGLQNSTITFWNITLEDEGCYMCLF |
| | | | 232 to | NTFGFGFYSGTACLTVYVQPIVSLHYKFSEDHLNITCSATARPAPMVFWKVPRSGIENSTVT |
| | | | amino acid | LSHPNGTTSVTSILHIKDPKNQVGKEVICQVLHLGTVTDFKQTVNKGERKCCVECPPCPAPP |
| | | | 1 of Fc | VAGPSVFLFPPKPKDTLMISRTPEVTCVVVDVSHEDPEVQFNWYVDGVEVHNAKTKPREE |
| | | | hinge | QFNSTFRVVSVLTVVHQDWLNGKEYKCKVSNKGLPAPIEKTISKTKGQPREPQVYTLPPSR |
| | | | | EEMTKNQVSLTCLVKGFYPSDISVEWESNGQPENNYKTTPPMLDSDGSFFLYSKLTVDKS |
| | | | | RWQQGNVFSCSVMHEALHNHYTQKSLSLSPGK (SEQ ID NO: 8) |
| DS-221 | K130Y | IgG2 | CD200 | MERLVIRMPFSHLSTYSLVWVMAAVVLCTAQVQVVTQDEREQLYTPASLKCSLQNAQEALI |
| | I131F | | amino acid | VTWQKKKAVSPENMVTFSENHGVVIQPAYKDKINITQLGLQNSTITFWNITLEDEGCYMCLF |
| | | | 232 to | NTFGFGYFSGTACLTVYVQPIVSLHYKFSEDHLNITCSATARPAPMVFWKVPRSGIENSTVT |
| | | | amino acid | LSHPNGTTSVTSILHIKDPKNQVGKEVICQVLHLGTVTDFKQTVNKGVECPPCPAPPVAGPS |
| | | | 6 of Fc | VFLFPPKPKDTLMISRTPEVTCVVVDVSHEDPEVQFNWYVDGVEVHNAKTKPREEQFNST |
| | | | hinge | FRVVSVLTVVHQDWLNGKEYKCKVSNKGLPAPIEKTISKTKGQPREPQVYTLPPSREEMTK |
| | | | | NQVSLTCLVKGFYPSDISVEWESNGQPENNYKTTPPMLDSDGSFFLYSKLTVDKSRWQQG |
| | | | | NVFSCSVMHEALHNHYTQKSLSLSPGK (SEQ ID NO: 9) |
| DS-149 | K130Y | IgG2 | CD200 | MERLVIRMPFSHLSTYSLVWVMAAVVLCTAQVQVVTQDEREQLYTPASLKCSLQNAQEALI |
| | I131F | | amino acid | VTWQKKKAVSPENMVTFSENHGVVIQPAYKDKINITQLGLQNSTITFWNITLEDEGCYMCLF |
| | | | 232 to | NTFGFGYFSGTACLTVYVQPIVSLHYKFSEDHLNITCSATARPAPMVFWKVPRSGIENSTVT |
| | | | amino acid | LSHPNGTTSVTSILHIKDPKNQVGKEVICQVLHLGTVTDFKQTVNKGERKCCVECPPCPAPP |
| | | | 1 of Fc | VAGPSVFLFPPKPKDTLMISRTPEVTCVVVDVSHEDPEVQFNWYVDGVEVHNAKTKPREE |
| | | | hinge | QFNSTFRVVSVLTVVHQDWLNGKEYKCKVSNKGLPAPIEKTISKTKGQPREPQVYTLPPSR |
| | | | | EEMTKNQVSLTCLVKGFYPSDISVEWESNGQPENNYKTTPPMLDSDGSFFLYSKLTVDKS |
| | | | | RWQQGNVFSCSVMHEALHNHYTQKSLSLSPGK (SEQ ID NO: 10) |
| DS-216 | I131F | IgG4 | CD200 | MERLVIRMPFSHLSTYSLVWVMAAVVLCTAQVQVVTQDEREQLYTPASLKCSLQNAQEALI |
| | | S228P | amino acid | VTWQKKKAVSPENMVTFSENHGVVIQPAYKDKINITQLGLQNSTITFWNITLEDEGCYMCLF |
| | | | 232 to | NTFGFGKFSGTACLTVYVQPIVSLHYKFSEDHLNITCSATARPAPMVFWKVPRSGIENSTVT |
| | | | amino acid | LSHPNGTTSVTSILHIKDPKNQVGKEVICQVLHLGTVTDFKQTVNKGPPCPPCPAPEFLGGP |
| | | | 6 of Fc | SVFLFPPKPKDTLMISRTPEVTCVVVDVSQEDPEVQFNWYVDGVEVHNAKTKPREEQFNS |
| | | | hinge | TYRVVSVLTVLHQDWLNGKEYKCKVSNKGLPSSIEKTISKAKGQPREPQVYTLPPSQEEMT |
| | | | | KNQVSLTCLVKGFYPSDIAVEWESNGQPENNYKTTPPVLDSDGSFFLYSRLTVDKSRWQE |
| | | | | GNVFSCSVMHEALHNHYTQKSLSLSLGK (SEQ ID NO: 11) |
| DS-214 | I131F | IgG4 | CD200 | MERLVIRMPFSHLSTYSLVWVMAAVVLCTAQVQVVTQDEREQLYTPASLKCSLQNAQEALI |
| | | S228P | amino acid | VTWQKKKAVSPENMVTFSENHGVVIQPAYKDKINITQLGLQNSTITFWNITLEDEGCYMCLF |
| | | | 232 to | NTFGFGKFSGTACLTVYVQPIVSLHYKFSEDHLNITCSATARPAPMVFWKVPRSGIENSTVT |
| | | | amino acid | LSHPNGTTSVTSILHIKDPKNQVGKEVICQVLHLGTVTDFKQTVNKGESKYGPPCPPCPAPE |
| | | | 1 of Fc | FLGGPSVFLFPPKPKDTLMISRTPEVTCVVVDVSQEDPEVQFNWYVDGVEVHNAKTKPRE |
| | | | hinge | EQFNSTYRVVSVLTVLHQDWLNGKEYKCKVSNKGLPSSIEKTISKAKGQPREPQVYTLPPS |
| | | | | QEEMTKNQVSLTCLVKGFYPSDIAVEWESNGQPENNYKTTPPVLDSDGSFFLYSRLTVDK |
| | | | | SRWQEGNVFSCSVMHEALHNHYTQKSLSLSLGK (SEQ ID NO: 12) |
| DS-174 | K130F | IgG4 | CD200 | MERLVIRMPFSHLSTYSLVWVMAAVVLCTAQVQVVTQDEREQLYTPASLKCSLQNAQEALI |
| | | S228P | amino acid | VTWQKKKAVSPENMVTFSENHGVVIQPAYKDKINITQLGLQNSTITFWNITLEDEGCYMCLF |
| | | | 232 to | NTFGFGFISGTACLTVYVQPIVSLHYKFSEDHLNITCSATARPAPMVFWKVPRSGIENSTVTL |
| | | | amino acid | SHPNGTTSVTSILHIKDPKNQVGKEVICQVLHLGTVTDFKQTVNKGPPCPPCPAPEFLGGPS |
| | | | 6 of Fc | VFLFPPKPKDTLMISRTPEVTCVVVDVSQEDPEVQFNWYVDGVEVHNAKTKPREEQFNST |
| | | | hinge | YRVVSVLTVLHQDWLNGKEYKCKVSNKGLPSSIEKTISKAKGQPREPQVYTLPPSQEEMTK |
| | | | | NQVSLTCLVKGFYPSDIAVEWESNGQPENNYKTTPPVLDSDGSFFLYSRLTVDKSRWQEG |
| | | | | NVFSCSVMHEALHNHYTQKSLSLSLGK (SEQ ID NO: 13) |
| DS-213 | K130F | IgG4 | CD200 | MERLVIRMPFSHLSTYSLVWVMAAVVLCTAQVQVVTQDEREQLYTPASLKCSLQNAQEALI |
| | | S228P | amino acid | VTWQKKKAVSPENMVTFSENHGVVIQPAYKDKINITQLGLQNSTITFWNITLEDEGCYMCLF |
| | | | 232 to | NTFGFGFISGTACLTVYVQPIVSLHYKFSEDHLNITCSATARPAPMVFWKVPRSGIENSTVTL |
| | | | amino acid | SHPNGTTSVTSILHIKDPKNQVGKEVICQVLHLGTVTDFKQTVNKGESKYGPPCPPCPAPEF |
| | | | 1 of Fc | LGGPSVFLFPPKPKDTLMISRTPEVTCVVVDVSQEDPEVQFNWYVDGVEVHNAKTKPREE |
| | | | hinge | QFNSTYRVVSVLTVLHQDWLNGKEYKCKVSNKGLPSSIEKTISKAKGQPREPQVYTLPPSQ |
| | | | | EEMTKNQVSLTCLVKGFYPSDIAVEWESNGQPENNYKTTPPVLDSDGSFFLYSRLTVDKSR |
| | | | | WQEGNVFSCSVMHEALHNHYTQKSLSLSLGK (SEQ ID NO: 14) |
| DS-163 | K130F | IgG4 | CD200 | MERLVIRMPFSHLSTYSLVWVMAAVVLCTAQVQVVTQDEREQLYTPASLKCSLQNAQEALI |
| | I131Y | S228P | amino acid | VTWQKKKAVSPENMVTFSENHGVVIQPAYKDKINITQLGLQNSTITFWNITLEDEGCYMCLF |
| | | | 232 to | NTFGFGFYSGTACLTVYVQPIVSLHYKFSEDHLNITCSATARPAPMVFWKVPRSGIENSTVT |
| | | | amino acid | LSHPNGTTSVTSILHIKDPKNQVGKEVICQVLHLGTVTDFKQTVNKGPPCPPCPAPEFLGGP |
| | | | 6 of Fc | SVFLFPPKPKDTLMISRTPEVTCVVVDVSQEDPEVQFNWYVDGVEVHNAKTKPREEQFNS |
| | | | hinge | TYRVVSVLTVLHQDWLNGKEYKCKVSNKGLPSSIEKTISKAKGQPREPQVYTLPPSQEEMT |
| | | | | KNQVSLTCLVKGFYPSDIAVEWESNGQPENNYKTTPPVLDSDGSFFLYSRLTVDKSRWQE |
| | | | | GNVFSCSVMHEALHNHYTQKSLSLSLGK (SEQ ID NO: 15) |
| DS-219 | K130F | IgG4 | CD200 | MERLVIRMPFSHLSTYSLVWVMAAVVLCTAQVQVVTQDEREQLYTPASLKCSLQNAQEALI |
| | I131Y | S228P | amino acid | VTWQKKKAVSPENMVTFSENHGVVIQPAYKDKINITQLGLQNSTITFWNITLEDEGCYMCLF |
| | | | 232 to | NTFGFGFYSGTACLTVYVQPIVSLHYKFSEDHLNITCSATARPAPMVFWKVPRSGIENSTVT |
| | | | amino acid | LSHPNGTTSVTSILHIKDPKNQVGKEVICQVLHLGTVTDFKQTVNKGESKYGPPCPPCPAPE |
| | | | 1 of Fc | FLGGPSVFLFPPKPKDTLMISRTPEVTCVVVDVSQEDPEVQFNWYVDGVEVHNAKTKPRE |
| | | | hinge | EQFNSTYRVVSVLTVLHQDWLNGKEYKCKVSNKGLPSSIEKTISKAKGQPREPQVYTLPPS |
| | | | | QEEMTKNQVSLTCLVKGFYPSDIAVEWESNGQPENNYKTTPPVLDSDGSFFLYSRLTVDK |
| | | | | SRWQEGNVFSCSVMHEALHNHYTQKSLSLSLGK (SEQ ID NO: 16) |
| DS-167 | K130F | IgG4 | CD200 | MERLVIRMPFSHLSTYSLVWVMAAVVLCTAQVQVVTQDEREQLYTPASLKCSLQNAQEALI |
| | I131Y | S228P | amino acid | VTWQKKKAVSPENMVTFSENHGVVIQPAYKDKINITQLGLQNSTITFWNITLEDEGCYMCLF |
| | | L235E | 232 to | NTFGFGFYSGTACLTVYVQPIVSLHYKFSEDHLNITCSATARPAPMVFWKVPRSGIENSTVT |
| | | P329G | amino acid | LSHPNGTTSVTSILHIKDPKNQVGKEVICQVLHLGTVTDFKQTVNKGPPCPPCPAPEFEGGP |
| | | | 6 of Fc | SVFLFPPKPKDTLMISRTPEVTCVVVDVSQEDPEVQFNWYVDGVEVHNAKTKPREEQFNS |
| | | | hinge | TYRVVSVLTVLHQDWLNGKEYKCKVSNKGLGSSIEKTISKAKGQPREPQVYTLPPSQEEMT |
| | | | | KNQVSLTCLVKGFYPSDIAVEWESNGQPENNYKTTPPVLDSDGSFFLYSRLTVDKSRWQE |
| | | | | GNVFSCSVMHEALHNHYTQKSLSLSLGK (SEQ ID NO: 17) |
| DS-165 | N95Q | IgG4 | CD200 | MERLVIRMPFSHLSTYSLVWVMAAVVLCTAQVQVVTQDEREQLYTPASLKCSLQNAQEALI |
| | N103Q | S228P | amino acid | VTWQKKKAVSPENMVTFSENHGVVIQPAYKDKIQITQLGLQQSTITFWQITLEDEGCYMCLF |
| | N110Q | | 232 to | NTFGFGFYSGTACLTVYVQPIVSLHYKFSEDHLQITCSATARPAPMVFWKVPRSGIEQSTVT |
| | K130F | | amino acid | LSHPQGTTSVTSILHIKDPKNQVGKEVICQVLHLGTVTDFKQTVNKGPPCPPCPAPEFLGGP |
| | I131Y | | 6 of Fc | SVFLFPPKPKDTLMISRTPEVTCVVVDVSQEDPEVQFNWYVDGVEVHNAKTKPREEQFNS |
| | N157Q | | hinge | TYRVVSVLTVLHQDWLNGKEYKCKVSNKGLPSSIEKTISKAKGQPREPQVYTLPPSQEEMT |
| | N181Q | | | KNQVSLTCLVKGFYPSDIAVEWESNGQPENNYKTTPPVLDSDGSFFLYSRLTVDKSRWQE |
| | N190Q | | | GNVFSCSVMHEALHNHYTQKSLSLSLGK (SEQ ID NO: 18) |
| DS-218 | K130F | IgG4 | CD200 | MERLVIRMPFSHLSTYSLVWVMAAVVLCTAQVQVVTQDEREQLYTPASLKCSLQNAQEALI |
| | I131F | S228P | amino acid | VTWQKKKAVSPENMVTFSENHGVVIQPAYKDKINITQLGLQNSTITFWNITLEDEGCYMCLF |
| | | | 232 to | NTFGFGFFSGTACLTVYVQPIVSLHYKFSEDHLNITCSATARPAPMVFWKVPRSGIENSTVT |
| | | | amino acid | LSHPNGTTSVTSILHIKDPKNQVGKEVICQVLHLGTVTDFKQTVNKGPPCPPCPAPEFLGGP |
| | | | 6 of Fc | SVFLFPPKPKDTLMISRTPEVTCVVVDVSQEDPEVQFNWYVDGVEVHNAKTKPREEQFNS |
| | | | hinge | TYRVVSVLTVLHQDWLNGKEYKCKVSNKGLPSSIEKTISKAKGQPREPQVYTLPPSQEEMT |
| | | | | KNQVSLTCLVKGFYPSDIAVEWESNGQPENNYKTTPPVLDSDGSFFLYSRLTVDKSRWQE |
| | | | | GNVFSCSVMHEALHNHYTQKSLSLSLGK (SEQ ID NO: 19) |
| DS-220 | K130F | IgG4 | CD200 | MERLVIRMPFSHLSTYSLVWVMAAVVLCTAQVQVVTQDEREQLYTPASLKCSLQNAQEALI |
| | I131F | S228P | amino acid | VTWQKKKAVSPENMVTFSENHGVVIQPAYKDKINITQLGLQNSTITFWNITLEDEGCYMCLF |
| | | | 232 to | NTFGFGFFSGTACLTVYVQPIVSLHYKFSEDHLNITCSATARPAPMVFWKVPRSGIENSTVT |
| | | | amino acid | LSHPNGTTSVTSILHIKDPKNQVGKEVICQVLHLGTVTDFKQTVNKGESKYGPPCPPCPAPE |
| | | | 1 of Fc | FLGGPSVFLFPPKPKDTLMISRTPEVTCVVVDVSQEDPEVQFNWYVDGVEVHNAKTKPRE |
| | | | hinge | EQFNSTYRVVSVLTVLHQDWLNGKEYKCKVSNKGLPSSIEKTISKAKGQPREPQVYTLPPS |
| | | | | QEEMTKNQVSLTCLVKGFYPSDIAVEWESNGQPENNYKTTPPVLDSDGSFFLYSRLTVDK |
| | | | | SRWQEGNVFSCSVMHEALHNHYTQKSLSLSLGK (SEQ ID NO: 20) |
| DS-222 | K130Y | IgG4 | CD200 | MERLVIRMPFSHLSTYSLVWVMAAVVLCTAQVQVVTQDEREQLYTPASLKCSLQNAQEALI |
| | I131F | S228P | amino acid | VTWQKKKAVSPENMVTFSENHGVVIQPAYKDKINITQLGLQNSTITFWNITLEDEGCYMCLF |
| | | | 232 to | NTFGFGYFSGTACLTVYVQPIVSLHYKFSEDHLNITCSATARPAPMVFWKVPRSGIENSTVT |
| | | | amino acid | LSHPNGTTSVTSILHIKDPKNQVGKEVICQVLHLGTVTDFKQTVNKGPPCPPCPAPEFLGGP |
| | | | 6 of Fc | SVFLFPPKPKDTLMISRTPEVTCVVVDVSQEDPEVQFNWYVDGVEVHNAKTKPREEQFNS |
| | | | hinge | TYRVVSVLTVLHQDWLNGKEYKCKVSNKGLPSSIEKTISKAKGQPREPQVYTLPPSQEEMT |
| | | | | KNQVSLTCLVKGFYPSDIAVEWESNGQPENNYKTTPPVLDSDGSFFLYSRLTVDKSRWQE |
| | | | | GNVFSCSVMHEALHNHYTQKSLSLSLGK (SEQ ID NO: 21) |
| DS-223 | K130Y | IgG4 | CD200 | MERLVIRMPFSHLSTYSLVWVMAAVVLCTAQVQVVTQDEREQLYTPASLKCSLQNAQEALI |
| | I131F | S228P | amino acid | VTWQKKKAVSPENMVTFSENHGVVIQPAYKDKINITQLGLQNSTITFWNITLEDEGCYMCLF |
| | | | 232 to | NTFGFGYFSGTACLTVYVQPIVSLHYKFSEDHLNITCSATARPAPMVFWKVPRSGIENSTVT |
| | | | amino acid | LSHPNGTTSVTSILHIKDPKNQVGKEVICQVLHLGTVTDFKQTVNKGESKYGPPCPPCPAPE |
| | | | 1 of Fc | FLGGPSVFLFPPKPKDTLMISRTPEVTCVVVDVSQEDPEVQFNWYVDGVEVHNAKTKPRE |
| | | | hinge | EQFNSTYRVVSVLTVLHQDWLNGKEYKCKVSNKGLPSSIEKTISKAKGQPREPQVYTLPPS |
| | | | | QEEMTKNQVSLTCLVKGFYPSDIAVEWESNGQPENNYKTTPPVLDSDGSFFLYSRLTVDK |
| | | | | SRWQEGNVFSCSVMHEALHNHYTQKSLSLSLGK (SEQ ID NO: 22) |

Specific examples of wild type CD200-Fc fusion proteins are exemplified in Table 2 as SEQ ID NOS: 23 to 25:

TABLE 2

| | | | | |
|---|---|---|---|---|
| DS-155 | Wild- | IgG4 | CD200 | MERLVIRMPFSHLSTYSLVWVMAAVVLCTAQVQVVTQDEREQLYTPASLKCSLQNAQEALIVT |
| | type | S228P | amino | WQKKKAVSPENMVTFSENHGVVIQPAYKDKINITQLGLQNSTITFWNITLEDEGCYMCLFNTFG |
| | | | acid 232 | FGYYSGTACLTVYVQPIVSLHYKFSEDHLNITCSATARPAPMVFWKVPRSGIENSTVTLSHPNG |
| | | | to amino | TTSVTSILHIKDPKNQVGKEVICQVLHLGTVTDFKQTVNKGPPCPPCPAPEFEGGPSVFLFPPK |
| | | | acid 6 of | PKDTLMISRTPEVTCVVVDVSQEDPEVQFNWYVDGVEVHNAKTKPREEQFNSTYRVVSVLTV |
| | | | Fc hinge | LHQDWLNGKEYKCKVSNKGLGSSIEKTISKAKGQPREPQVYTLPPSQEEMTKNQVSLTCLVK |
| | | | | GFYPSDIAVEWESNGQPENNYKTTPPVLDSDGSFFLYSRLTVDKSRWQEGNVFSCSVMHEAL |
| | | | | HNHYTQKSLSLSLGK (SEQ ID NO: 23) |
| DS-156 | Wild- | IgG2 | CD200 | MERLVIRMPFSHLSTYSLVWVMAAVVLCTAQVQVVTQDEREQLYTPASLKCSLQNAQEALIVT |
| | type | | amino | WQKKKAVSPENMVTFSENHGVVIQPAYKDKINITQLGLQNSTITFWNITLEDEGCYMCLFNTFG |
| | | | acid 232 | FGKISGTACLTVYVQPIVSLHYKFSEDHLNITCSATARPAPMVFWKVPRSGIENSTVTLSHPNG |
| | | | to amino | TTSVTSILHIKDPKNQVGKEVICQVLHLGTVTDFKQTVNKGVECPPCPAPPVAGPSVFLFPPKP |
| | | | acid 6 of | KDTLMISRTPEVTCVVVDVSHEDPEVQFNWYVDGVEVHNAKTKPREEQFNSTFRVVSVLTVV |
| | | | Fc hinge | HQDWLNGKEYKCKVSNKGLPAPIEKTISKTKGQPREPQVYTLPPSREEMTKNQVSLTCLVKGF |
| | | | | YPSDISVEWESNGQPENNYKTTPPMLDSDGSFFLYSKLTVDKSRWQQGNVFSCSVMHEALH |
| | | | | NHYTQKSLSLSPGK (SEQ ID NO: 24) |
| DS-226 | Wild- | IgG2 | CD200 | MERLVIRMPFSHLSTYSLVWVMAAVVLCTAQVQVVTQDEREQLYTPASLKCSLQNAQEALIVT |
| | type | | amino | WQKKKAVSPENMVTFSENHGVVIQPAYKDKINITQLGLQNSTITFWNITLEDEGCYMCLFNTFG |
| | | | acid 232 | FGKISGTACLTVYVQPIVSLHYKFSEDHLNITCSATARPAPMVFWKVPRSGIENSTVTLSHPNG |
| | | | to amino | TTSVTSILHIKDPKNQVGKEVICQVLHLGTVTDFKQTVNKGERKCCVECPPCPAPPVAGPSVFL |
| | | | acid 1 of | FPPKPKDTLMISRTPEVTCVVVDVSHEDPEVQFNWYVDGVEVHNAKTKPREEQFNSTFRVVS |
| | | | Fc hinge | VLTVVHQDWLNGKEYKCKVSNKGLPAPIEKTISKTKGQPREPQVYTLPPSREEMTKNQVSLTC |
| | | | | LVKGFYPSDISVEWESNGQPENNYKTTPPMLDSDGSFFLYSKLTVDKSRWQQGNVFSCSVM |
| | | | | HEALHNHYTQKSLSLSPGK (SEQ ID NO: 25) |

The proteins of the present invention are preferably produced by recombinant DNA methods by inserting a nucleic acid sequence encoding mutated CD200 protein or any portion thereof into a recombinant expression vector and expressing the nucleic acid sequence in a recombinant expression system under conditions promoting expression. Therefore, in one embodiment, the polynucleotide encoding the fusion protein additionally comprises a vector, such as pcDNA 3.4. In one embodiment, the fusion protein is flanked by one or more restriction enzyme sites, such as Hind III and/or Xho I. In a further embodiment, the polynucleotide encoding the fusion protein is flanked by Hind III and Xho I restriction sites.

In one embodiment, the fusion protein comprises one or more restriction enzyme sites, such as Bam HI.

According to a further aspect of the invention, there is provided a polynucleotide encoding a protein as defined herein and use of such nucleic acids to produce the proteins and/or for therapeutic purposes. Such polynucleotides may include DNA and RNA molecules (e.g., mRNA, self-replicating RNA, self-amplifying mRNA, etc.) that encode a protein as defined herein. Nucleic acid sequences encoding the proteins provided by this invention can be assembled from cDNA fragments and short oligonucleotide linkers, or from a series of oligonucleotides, to provide a synthetic gene which is capable of being inserted in a recombinant expression vector and expressed in a recombinant transcriptional unit.

Recombinant expression vectors include synthetic or cDNA-derived nucleic acid fragments encoding mutated CD200 operably linked to suitable transcriptional or translational regulatory elements derived from mammalian, microbial, viral or insect genes. Such regulatory elements include a transcriptional promoter, an optional operator sequence to control transcription, a sequence encoding suitable mRNA ribosomal binding sites, and sequences which control the termination of transcription and translation. The ability to replicate in a host, usually conferred by an origin of replication, and a selection gene to facilitate recognition of transformants may additionally be incorporated.

### Therapeutic Uses

The invention has particular application in therapy because the interaction between the CD200 protein and the CD200 receptor is characterized by rapid dissociation (“off”) rates which results in low affinity of CD200 for the CD200 receptor. Therefore, increasing the affinity of mutant CD200 protein for the CD200 receptor as presented herein, can be used in the manufacture of pharmaceutical compositions with more potent properties.

Furthermore, manufacturing costs for recombinant proteins are high and the mutant CD200 protein, having higher affinity, can be used in pharmaceutical compositions at significantly lower doses than wild type or non-mutated CD200 protein to achieve a therapeutic effect. Use of the mutant CD200 protein may therefore be more cost effective in addition to being more clinically effective.

According to a further aspect of the invention, there is provided a pharmaceutical composition comprising the protein, polypeptide or fusion protein or a nucleic acid encoding the protein, polypeptide or fusion protein as defined herein.

In one embodiment, the mutated CD200 protein, polypeptide or fusion protein as defined herein is a modulator of the CD200 receptor. The term “modulator” as used herein, refers to a substance which results in a change, for example a modulator of a protein may result in an increase or decrease in the activity of said protein. In view of the properties of the mutated CD200 proteins and fusion proteins of the invention, they are believed to be agonists of the CD200 receptor and therefore find utility in the treatment of autoimmune disease. Therefore, in a further embodiment, the mutated CD200 protein, polypeptide or fusion protein as defined herein is an agonist of the CD200 receptor.

Thus, according to a further aspect of the invention, there is provided the protein, polypeptide or fusion protein as defined herein or the composition as defined herein for use in the treatment of an autoimmune disease.

As used herein, the terms “autoimmune disease” or “autoimmune disorder” are used interchangeably and refer to undesirable conditions that arise from an inappropriate or unwanted immune reaction against self-cells and/or tissues or transplanted cells and/or tissues. The term “autoimmune disease” or “autoimmune disorder” is meant to include such conditions, whether they be mediated by humoral or cellular immune responses.

In an alternative embodiment, there is provided the protein, polypeptide or fusion protein as defined herein or the composition as defined herein for use in the treatment of an allergic disease. As used herein, the terms “allergy” or “allergic disease” are used interchangeably and refer to a T helper 2 (TH2)-driven disease that develops primarily from activity of TH2 cells. Examples of allergic diseases include chronic allergic disease (such as hay fever or allergic rhinitis), allergic contact dermatitis, seasonal allergies, anaphylaxis treatment and prevention and food allergies.

Fusion proteins comprising the mutant CD200 proteins defined herein may deactivate activated immune cells with higher efficiency than fusion proteins comprising wild-type or non-mutated CD200 proteins.

In one embodiment, the autoimmune disease is selected from autoimmune diseases affecting the neuromuscular system, vascular system, eye, digestive tract, lung, kidney, liver, peripheral or central nervous system, bone, cartilage or joints.

In a further embodiment, the autoimmune disease is one or more autoimmune diseases selected from: acute disseminated encephalomyelitis (ADEM); acute necrotizing haemorrhagic leukoencephalitis; Addison's disease; agammaglobulinemia; alopecia areata; amyloidosis; ankylosing spondylitis; anti-GBM/anti-TBM nephritis; antiphospholipid syndrome (APS); asthma, atopic dermatitis; Autoimmune angioedema; autoimmune aplastic anemia; autoimmune dysautonomia; autoimmune hepatitis; autoimmune hyperlipidemia; autoimmune immunodeficiency; autoimmune inner ear disease (AIED); autoimmune myocarditis; autoimmune oophoritis; autoimmune pancreatitis; autoimmune retinopathy; autoimmune thrombocytopenia purpura (ATP); autoimmune thyroid disease; autoimmune urticarial; axonal & neuronal neuropathies; Balo disease; Behcet's disease; bullous pemphigoid and related autoimmune blistering diseases; cardiomyopathy; Castleman disease; celiac disease (such as refractory celiac disease type II); Chagas disease; chronic idiopathic urticaria; chronic inflammatory demyelinating polyneuropathy (CIDP); chronic obstructive pulmonary disease (COPD); chronic recurrent multifocal osteomyelitis (CRMO); chronic spontaneous urticaria; Churg-Strauss syndrome; cicatricial pemphigoid/benign mucosal pemphigoid; Crohn's disease; Cogans syndrome; cold agglutinin disease; congenital heart block; Coxsackie myocarditis; CREST disease; essential mixed cryoglobulinemia; demyelinating neuropathies; dermatitis herpetiformis; dermatomyositis; Devic's disease (neuromyelitis optica); discoid lupus; Dressler's syndrome; endometriosis; eosinophilic esophagitis; eosinophilic fasciitis; erythema nodosum; experimental allergic encephalomyelitis; Evans syndrome; fibrosing alveolitis; giant cell arteritis (temporal arteritis); giant cell myocarditis; glomerulonephritis; Goodpasture's syndrome; granulomatosis with polyangiitis (GPA) (formerly called Wegener's granulomatosis); graft-versus-host disease (GvHD); Graves' disease; Guillain-Barre syndrome; Hashimoto's encephalitis; Hashimoto's thyroiditis; hemolytic anemia; Henoch-Schonlein purpura; herpes gestationis; hypogammaglobulinemia; idiopathic thrombocytopenia purpura (ITP); IgA nephropathy; IgG4-related sclerosing disease; immunoregulatory lipoproteins; inclusion body myositis; inflammatory bowel disorder (IBD); inflammatory skin disease; interstitial cystitis; juvenile arthritis; juvenile diabetes (type 1 diabetes); juvenile myositis; Kawasaki syndrome; Lambert-Eaton syndrome; leukocytoclastic vasculitis; lichen planus; lichen sclerosus; ligneous conjunctivitis; linear IgA disease (LAD); systemic lupus erythematosus (SLE); lyme disease, chronic; macrophage activation syndrome (MAS); mastocytosis; Meniere's disease; microscopic polyangiitis; mixed connective tissue disease (MCTD); Mooren's ulcer; Mucha-Habermann disease; multiple sclerosis; myasthenia gravis; myositis; narcolepsy; neuromyelitis optica (Devic's); neutropenia; ocular cicatricial pemphigoid; optic neuritis; palindromic rheumatism; PANDAS (Pediatric Autoimmune Neuropsychiatric Disorders Associated with Streptococcus); paraneoplastic cerebellar degeneration; paroxysmal nocturnal hemoglobinuria (PNH); Parry Romberg syndrome; Parsonnage-Turner syndrome; pars planitis (peripheral uveitis); pemphigus; peripheral neuropathy; perivenous encephalomyelitis; pernicious anemia; POEMS syndrome; polyarteritis nodosa; type I, II, & III autoimmune polyglandular syndromes; polymyalgia rheumatic; polymyositis; postmyocardial infarction syndrome; postpericardiotomy syndrome; progesterone dermatitis; primary biliary cirrhosis; primary sclerosing cholangitis; psoriasis; psoriatic arthritis; idiopathic pulmonary fibrosis; pyoderma gangrenosum; pure red cell aplasia; Raynauds phenomenon; reactive arthritis; reflex sympathetic dystrophy; Reiter's syndrome; relapsing polychondritis; restless legs syndrome; retroperitoneal fibrosis; rheumatic fever; rheumatoid arthritis; sarcoidosis; Schmidt syndrome; scleritis; scleroderma; Sjogren's syndrome; sperm & testicular autoimmunity; stiff person syndrome; subacute bacterial endocarditis (SBE); Susac's syndroms; sympathetic ophthalmia; Takayasu's arteritis; temporal arteritis/giant cell arteritis; thrombocytopeniarpura (TTP); Tolosa-Hunt syndrome; transverse myelitis; type 1 diabetes; ulcerative colitis; undifferentiated connective tissue disease (UCTD); uveitis; vasculitis; vesiculobullous dermatosis; vitiligo; and Wegener's granulomatosis (now termed granulomatosis with polyangiitis (GPA).

In a yet further embodiment, the autoimmune disease is one or more autoimmune diseases selected from: atopic dermatitis, alopecia areata, asthma, systemic lupus erythematosus (SLE), inflammatory bowel disorder (IBD), chronic obstructive pulmonary disease (COPD), multiple sclerosis, and rheumatoid arthritis.

In an alternative embodiment, there is provided the protein, polypeptide or fusion protein as defined herein or the composition as defined herein for use in the treatment of neurodegeneration.

In an alternative embodiment, there is provided the protein, polypeptide or fusion protein as defined herein or the composition as defined herein for use in the treatment of neuropathic pain. In a further embodiment, there is provided the protein, polypeptide or fusion protein as defined herein or the composition as defined herein for use in the treatment of neuropathic pain, such as diabetic neuropathy.

According to a further aspect of the invention, there is provided a method of treating an autoimmune disease, an allergic disease, neurodegeneration or neuropathic pain in a subject, comprising administering a protein, polypeptide or fusion protein of the invention to a subject having at least one autoimmune disease, allergic disease, neurodegeneration or neuropathic pain.

It will be appreciated that a protein, polypeptide or fusion protein of the invention can be administered as the sole therapeutic agent or it can be administered in combination therapy with one of more other compounds (or therapies) for the treatment of an autoimmune disease, an allergic disease, neurodegeneration or neuropathic pain.

Thus, according to a further aspect of the invention there is provided a pharmaceutical composition comprising a protein, polypeptide or fusion protein as defined herein in combination with one or more additional therapeutic agents.

For the treatment of an autoimmune disease, an allergic disease, neurodegeneration or neuropathic pain, the protein, polypeptide or fusion protein of the invention may be advantageously employed in combination with one or more other medicinal agents, more particularly, with one or more immunosuppressive agents or adjuvants in immunosuppression therapy.

Examples of other therapeutic agents or treatments that may be administered together (whether concurrently or at different time intervals) with the compounds of the invention include but are not limited to: azathioprine; methotrexate; cyclosporine; monoclonal antibodies (basiliximab, daclizumab, and muromonab); and corticosteroids.

Each of the therapeutic agents present in the combinations of the invention may be given in individually varying dose schedules and via different routes. Additionally, the posology of each of the two or more agents may differ: each may be administered at the same time or at different times. A person skilled in the art would know through his or her common general knowledge the dosing regimens and combination therapies to use. For example, a protein, polypeptide or fusion protein of the invention may be used in combination with one or more other agents which are administered according to their existing combination regimen.

Generally, the proteins disclosed herein will be utilised in purified form together with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically acceptable adjuvants, if necessary to keep a polypeptide complex in suspension, may be chosen from thickeners such as carboxymethylcellulose, polyvinylpyrrolidone, gelatin and alginates.

The route of administration of pharmaceutical compositions according to the invention may be any of those commonly known to those of ordinary skill in the art. For therapy, including without limitation immunotherapy, the proteins of the invention can be administered to any patient in accordance with standard techniques. The administration can be by any appropriate mode, including parenterally, intravenously, intramuscularly, intraperitoneally, subcutaneously, transdermally, via the pulmonary route, or also, appropriately, by direct infusion with a catheter. The dosage and frequency of administration will depend on the age, sex and condition of the patient, concurrent administration of other drugs, contraindications and other parameters to be taken into account by the clinician.

The proteins of the invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that levels may have to be adjusted upward to compensate.

The following studies and protocols illustrate embodiments of the methods described herein.

### Example 1: Manufacture of Mutant and Wild Type CD200-Fc Molecules

### Gene Synthesis

A DNA sequence encoding mutant or wild-type human CD200 residues 1-232 of Uniprot P412178 (OX2G_Human), which includes an N-terminal signal sequence, was fused at the C-terminus to mutant or wild-type IgG2 or IgG4 Fc with direct fusion of amino acid Glycine 232 of CD200 to amino acid 1 of the Fc hinge region, or to amino acid 6 of the Fc hinge region (in the latter case deleting the first 5 amino acids of the Fc hinge). Gene synthesis was carried out at GeneArt for the wild-type and mutant constructs.

The expression constructs were made using the mammalian expression vector pcDNA3.4, with 5′ Hind Ill and 3′Xho. An internal Bam HI was introduced to facilitate Fc domain swapping.

### Midi Prep

Upon receipt, the lyophilized DNA constructs of the CD200-Fc target proteins (both wild-type and mutant CD200-Fc proteins) were suspended in 50 μl of MQ and transformed DH 5a cells. A single colony of each target protein was selected and inoculated into 5.0 ml of LB containing ampicillin. Next, DNA from 2.0 ml of culture was isolated for confirmation and resolved by Agarose gel electrophoresis. The constructs were confirmed by digesting the DNA with Hind and XhoI. Each mutant or wild-type construct was cultured into 100 ml of LB for the midi scale DNA preparation. The DNA was isolated using the purelink Hipure plasmid midiprep kit.

### Protein Expression

The CD200-Fc target proteins were manufactured using the Thermo Fisher Gibco™ ExpiCHO™ expression system according to the manufacturer's instructions for a 25 ml culture volume. The media supernatant containing the expressed CD200-Fc was collected and stored at −80° C. until use.

### Protein Purification

Buffer exchange was performed using a Hiprep desalting column (XK26/10) packed with 53 ml of SephadexG25 to condition the media for affinity column purification. The desalting columns were equilibrated with Buffer A (150 mM NaCl containing 20 mM Sodium phosphate pH 7.4) on an AKTA explorer platform. 30 ml of clarified culture media was loaded onto two desalting columns connected in series at a rate of 1 ml/min. The protein was eluted at a rate of 2 ml/min and collected in fractions. Fractions showing a maximum absorbance and pH 7.4-7.2 were pooled. Fractions exhibiting a lower pH were rejected. Following the desalting the samples were diluted to make up approximately 45 ml of wild-type or mutant CD200-Fc supernatant. All of the purification procedures were performed on ice at 4° C.

The column was washed with 10 column volumes of Buffer A (10 ml of 20 mM sodium phosphate pH 7.4, 150 mM NaCl). CD200-Fc Protein was eluted with a pH 7.4-3.5 gradient over 10-column volumes using 20 mM Sodium Phosphate pH 7.4, 150 mM NaCl and 100 mM Citrate buffer pH 3.5 in a linear gradient. The CD200-Fc fractions comprising the dimeric form of the protein (calculated to be approximately 103 kDa) were collected. The protein buffer was exchanged using an Amicon ultra centricon with a 10 kDa cut-off and the protein concentrated to around 1 mg/ml.

### Example 2: Binding Analysis of the Wild-Type and Mutant CD200-Fc Molecules

Biacore experiments were performed by Syngene International Ltd. (Biocon Park, Plot No 2&3, Bommasandra Industrial Area, Bommasadra-Jigani Link Road, Bangalore-560099, India).

### Assay Principles

BIAcore instrumentation uses an optical method, Surface Plasmon Resonance (SPR), to measure the binding characteristics of two interacting molecules; in this case wild-type CD200-Fc or CD200-Fc mutants binding to the CD200 receptor (CD200R). The technique measures changes in the refractive index of one of the two interacting molecules captured on a chip (sensor) when the second molecule is flowed in solution over the captured partner. In these experiments CD200-Fc was immobilized on the chip (sensor) surface and CD200R was injected in an aqueous buffer over the captured CD200-Fc under continuous flow conditions. Changes in the CD200-Fc refractive index following CD200R binding were measured in real time and the result plotted as response units (Rus) versus time to generate sensorgrams.

### Instrumentation and Reagents

The experiments were performed on a GE Healthcare BIAcore T200. Table 3 details the reagents used in developing and performing the assay.

TABLE 3
Reagents used in the course of the BIAcore experiments

| SI. No. | Product Description | Vendor | Catalogue No. |
|---|---|---|---|
| 1 | Human Antibody Capture Kit | GE Healthcare | BR1008-39 |
| 2 | Recombinant HumanCD200R, | Creative | CD200R1- |
| | His tagged | Biomart | 320H |
| 3 | Recombinant HumanCD200, | Creative | CD200-165H |
| | Fc-tagged | Biomart | |
| 4 | Recombinant Mouse CD200 | Creative | CD200-982M |
| | Protein, Fc Chimera | Biomart | |
| 5 | Trastuzumab | Roche | N/A |

### CD200-Fc Immobilization

Anti-human Fc was covalently immobilized on a BIAcore CM5 sensor chip by amine coupling using a GE Healthcare kit following the manufacturer's instructions. Maximum immobilization target was set between 10000-15000 RU. Flow cell 1 was used as a reference, with no immobilized ligand, to permit deduction of non-specific binding to the chip surface. The Fc-ligands were diluted to 0.5 μg/mL in BIAcore running buffer (HBS-EP+: 10 mM HEPES buffered saline containing 2 mM EDTA and 0.05% surfactant P-20). In the final immobilization step, wild-type CD200-Fc, mutant CD200-Fc and un-related control protein (Herceptin/Trastuzumab) were passed over the chip (using flow cells 2, 3 and 4 respectively), for 120 seconds, at a concentration giving rise to a minimum of 250 response units (RU), followed by stabilization of the surface for 120 seconds in running buffer. The CD200-Fc capture procedure was repeated for every CD200R concentration.

### Passage of CD200R Over the CD200-Fc-Bound Chip Surface

Following capture of the Fc-tagged proteins, CD200R (at different concentrations) was flowed over the captured CD200-Fc and control proteins for 120 seconds (to observe association), followed by 120 seconds of running buffer (to observe dissociation). The chip surface was then regenerated using 10 mM Glycine-HCl (pH 2) for 30 seconds (30 μl/min flow rate) followed by stabilization of the surface for 60 seconds with BIAcore running buffer before the next cycle. All CD200R concentrations were run in duplicate at the following concentrations: 1 μM, 500 nM, 250 nM, 125 nM, 62.5 nM, 31.25 nM, 15.6 nM and 0 nM.

### Data Analysis

The results were represented in sensorgrams plotted as response or resonance units (Rus) versus time. The experimental sensorgrams were analyzed in BIAevaluation software version 1.0 (GE Healthcare). Curve fitting for all the CD200-Fc fusion molecules except DS-162 was carried out using a 1:1 Langmuir binding model. The DS-162 curves did not fit well to a 1:1 binding model. Both 1:1 and two state binding models were used to fit the DS-162 results. The dissociation phase of the DS-162 sensorgram was found to fit the two-state binding model better than the 1:1 binding model (FIG. 1, panels E-F). Rate equations using standard parameters (e.g. ligand concentration, time) were used for iterative curve fitting. Closeness of fit was determined by algorithms provided by the manufacturer in the BIAevaluation software version 1.0.

### Results

The results (Table 4 and FIG. 1) show that the mutated CD200-Fc proteins bind to the human CD200 receptor with 16-70 fold greater affinity than wild-type CD200-Fc. The tabulated off rates in Table 4 and the sensorgrams illustrated in FIG. 1 exhibit a range of off rates and half lives on the receptor. In particular DS-161 and DS-162 combine high affinity binding to the CD200 receptor with off rates compatible with efficient agonism in functional cellular assays.

TABLE 4
Surface Plasmon Resonance (SPR) affinity (K_{D}) and kinetic parameters
(kₐ, k_{D}, t½) of wild-type and engineered CD200-Fc
fusion molecules for the human CD200 Receptor at 25° C.

| | | | ka | kD | KD | t½ |
|---|---|---|---|---|---|---|
| ID | Mutation | Fc | (1/Ms) | (1/s) | (nM) | (s) |
| DS-155 | Wild-Type | IgG4 | 5.43E+05 | 2.51E−02 | 50 | 28 |
| | | S228P | | | | |
| DS-161 | K130F | IgG2 | 8.38E+05 | 5.93E−04 | 0.7 | 1169 |
| DS-162¹ | I131F | IgG2 | 1.90E+06 | 4.74E−03 | 3.0 | 146 |
| DS-162² | I131F | IgG2 | 2 rates | 2 rates | 3.0 | No |
| | | | calculated | calculated | | single |
| | | | | | | t½ |
| DS-150 | K130F, | IgG2 | 8.75E+04 | 2.26E−04 | 2.6 | 3073 |
| | I131F | | | | | |
| DS-149 | K130Y, | IgG2 | 131500 | 0.000228501 | 1.9 | 3033 |
| | I131F | | | | | |
| DS-151 | K130F, | IgG2 | 1.28E+05 | 1.81E−04 | 1.4 | 3829 |
| | I131Y | | | | | |

¹1:1 binding model
²two state binding model

### Example 3: Inhibition of IL-6 Secretion Following LPS Stimulation of CD200R Expressing U937 Cells

To demonstrate the agonist activity of the CD200-Fc proteins the human monocyte cell line U937 (ATCC, CRL1539) was transfected with the cDNA for human CD200R. Cytokine production, including IL-6, from these cells can be induced by stimulation with PMA and then LPS.

50,000 U937 cells per well were seeded in 96 well plates and differentiated following incubation for 72 hours with 100 nM PMA. Following differentiation, the PMA containing media was replaced with fresh assay media and incubated for a further 2 hours prior to treatment. CD200-Fc constructs (including DS-226 as wild type control) were then added to the cell culture and incubated for one hour. Cells were stimulated with 100 ng/ml LPS and incubated for a further twenty four hours. Following the final incubation cell supernatant (diluted 1:10) was collected and IL-6 secretion quantified using an ELISA assay.

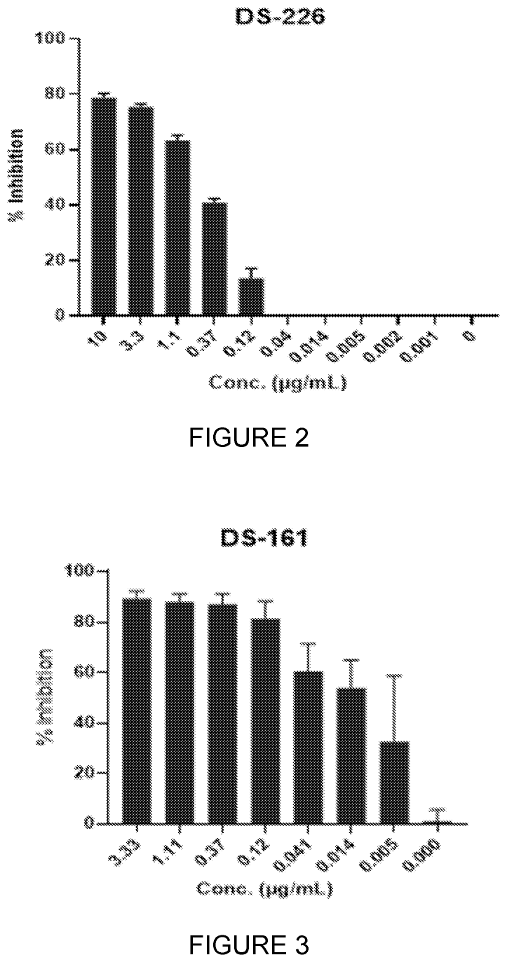

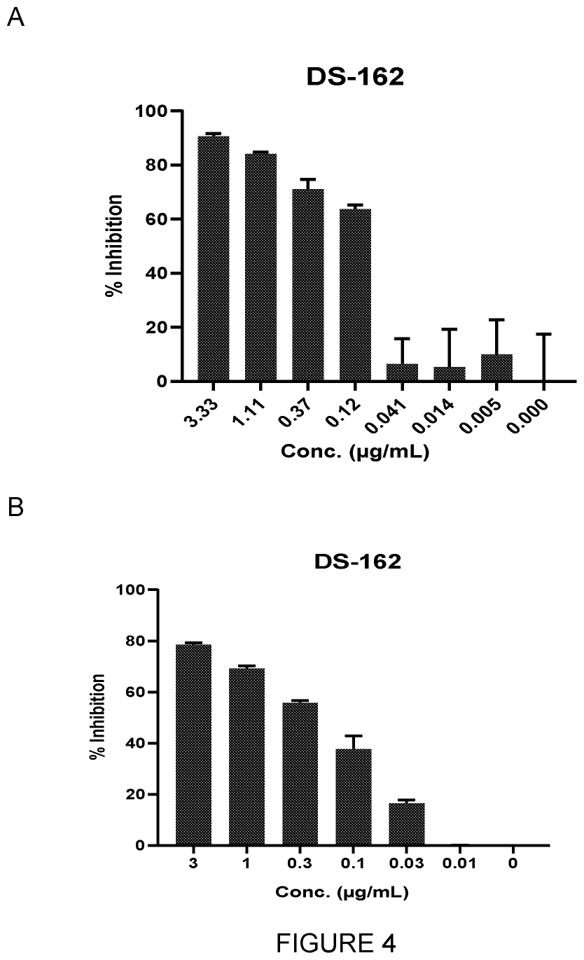

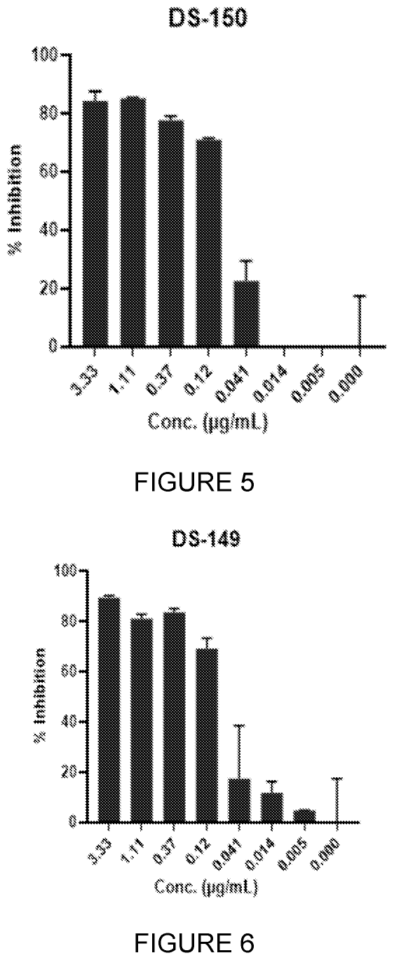

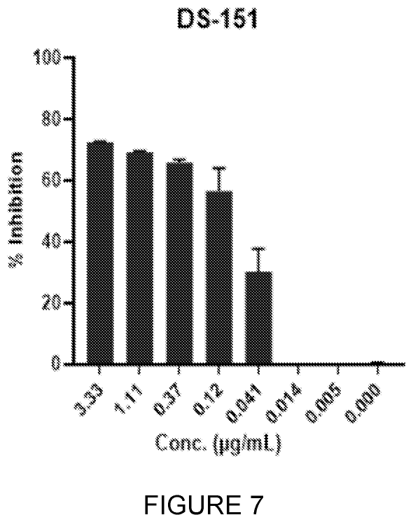

FIGS. 2 to 7 illustrate the concentration dependent inhibition of IL-6 secretion by the CD200-Fc constructs.

The data shown demonstrate that the CD200-Fc constructs are able to inhibit LPS stimulated IL-6 secretion in a concentration dependent manner.

1. A mutated CD200 protein comprising the following mutations:
(i) K130F; or
(ii) I131F; or
(iii) K130F and I131F; or
(iv) K130F and I131Y; or
(v) K130Y and I131F.

2. A polypeptide comprising a mutated CD200 protein comprising at least 90% identity to:
(SEQ ID NO: 26)
QVQVVTQDEREQLYTPASLKCSLQNAQEALIVTWQKKKAVSPENMVTFS
ENHGVVIQPAYKDKINITQLGLQNSTITFWNITLEDEGCYMCLFNTFGF
GKISGTACLTVYVQPIVSLHYKFSEDHLNITCSATARPAPMVFWKVPRS
GIENSTVTLSHPNGTTSVTSILHIKDPKNQVGKEVICQVLHLGTVTDFK
QTVNK
with the following mutations at positions 130 and/or 131:
(i) K130F; or
(ii) I131F; or
(iii) K130F and I131F; or
(iv) K130F and I131Y; or
(v) K130Y and I131F.

3. The polypeptide of claim 2, comprising at least 90% identity to the amino acid sequence of:
(SEQ ID NO: 27)
MERLVIRMPFSHLSTYSLVWVMAAVVLCTAQVQVVTQDEREQLYTPASL
KCSLQNAQEALIVTWQKKKAVSPENMVTFSENHGVVIQPAYKDKINITQ
LGLQNSTITFWNITLEDEGCYMCLFNTFGFGKISGTACLTVYVQPIVSL
HYKFSEDHLNITCSATARPAPMVFWKVPRSGIENSTVTLSHPNGTTSVT
SILHIKDPKNQVGKEVICQVLHLGTVTDFKQTVNKGYWFSVPLLLSIVS
LVILLVLISILLYWKRHRNQDRGELSQGVQKMT
with the following mutations at positions 130 and/or 131:
(i) K130F; or
(ii) I131F; or
(iii) K130F and I131F; or
(iv) K130F and I131Y; or
(v) K130Y and I131F.

4. A fusion protein comprising the protein or polypeptide as defined in claim 1, fused to a non-CD200 portion directly or via a linker portion.

5. The fusion protein as defined in claim 4, wherein said non-CD200 portion is an antibody or fragment thereof.

6. The fusion protein as defined in claim 5, wherein said non-CD200 portion is an Fc fragment.

7. The fusion protein as defined in claim 6, wherein said Fc fragment is or is derived from human IgG2 or human IgG4, such as a S228P derivative of human IgG4.

8. The fusion protein as defined in claim 6, which is an Fc fusion protein formed by direct fusion of amino acid Glycine 232 of CD200 to amino acid 1 of the Fc hinge region, or an Fc fusion protein formed by direct fusion of amino acid Glycine 232 of CD200 to amino acid 6 of the Fc hinge region.

9. The fusion protein as defined in claim 4, which is selected from any one of SEQ ID NOS: 1 to 22.

10. The protein of, claim 1, which is a modulator of the CD200 receptor.

11. The protein of, claim 1, which is an agonist of the CD200 receptor.

12. A polynucleotide encoding a protein, the protein of claim 1.

13. A pharmaceutical composition comprising the protein of, claim 1, and a carrier.

14. (canceled)

15. (canceled)

16. (canceled)

17. A method of treating a subject having an autoimmune disease, an allergic disease, neurodegeneration, or neuropathic pain comprising administering the protein of claim 1 to the subject.

18. The method as defined in claim 17 wherein the autoimmune disease is selected from: atopic dermatitis, alopecia areata, asthma, systemic lupus erythematosus (SLE), inflammatory bowel disorder (IBD), chronic obstructive pulmonary disease (COPD), multiple sclerosis, and rheumatoid arthritis.

19. The method as defined in claim 17 wherein the neuropathic pain is diabetic neuropathy.

20. The fusion protein of claim 8, wherein the mutated CD200 protein has mutation K130F or I131F.

21. A method of treating a subject having an autoimmune disease, an allergic disease, neurodegeneration, or neuropathic pain comprising administering the polypeptide of claim 2 to the subject.

22. A method of treating a subject having an autoimmune disease, an allergic disease, neurodegeneration, or neuropathic pain comprising administering the polypeptide of claim 3 to the subject.

23. A method of treating a subject having an autoimmune disease, an allergic disease, neurodegeneration, or neuropathic pain comprising administering the fusion protein of claim 8 to the subject.
